SEQUENCE LISTING (17 sequences)

SEQ ID NO: 1 - protein, 4 aa, Artificial
  synthetic proteosome inhibitor with binding affinity for human proteins involved in eNOS regulation
IGAL

SEQ ID NO: 2 - protein, 4 aa, Artificial
  synthetic proteosome inhibitor with binding affinity for human proteins involved in eNOS regulation
YLLL

SEQ ID NO: 3 - protein, 4 aa, Artificial
  synthetic proteosome inhibitor with binding affinity for human proteins involved in eNOS regulation
YLLL

SEQ ID NO: 4 - protein, 4 aa, Artificial
  synthetic proteosome inhibitor with binding affinity for human proteins involved in eNOS regulation
KLLL

SEQ ID NO: 5 - protein, 4 aa, Artificial
  synthetic proteosome inhibitor with binding affinity for human proteins involved in eNOS regulation
IITL

SEQ ID NO: 6 - DNA, 21 bp, Homo sapiens
ggcatcaccaggaagaagacc

SEQ ID NO: 7 - DNA, 19 bp, Homo sapiens
ttcactcgcttcgccatca

SEQ ID NO: 8 - DNA, 29 bp, Homo sapiens
taaagaagtggccaacgccgtgaagatct

SEQ ID NO: 9 - DNA, 25 bp, Homo sapiens
agtctggcttatatccaacacttcg

SEQ ID NO: 10 - DNA, 22 bp, Homo sapiens
gactttgctttccttggtcagg

SEQ ID NO: 11 - DNA, 26 bp, Homo sapiens
tttcaccagcaagcttgcgaccttga

SEQ ID NO: 12 - DNA, 28 bp, Bos taurus
tttaccataagagactggaccagaagtt

SEQ ID NO: 13 - DNA, 22 bp, Bos taurus
attgacagcactggcttaggca

SEQ ID NO: 14 - DNA, 25 bp, Bos taurus
gctataagttctttgccgacctgtt

SEQ ID NO: 15 - DNA, 26 bp, Bos taurus
ttctgttcagtgctttgatgtaatcc

SEQ ID NO: 16 - DNA, 22 bp, Bos taurus
agtagctggttccctccgaagt

SEQ ID NO: 17 - DNA, 19 bp, Bos taurus
ttgcgagagcgccagctat

(19) United States
(12) Patent Application Publication
(10) Pub. No.: US 2006/0199772 A1
(43) Pub. Date: Sep. 7, 2006

(54) Use of a proteasome inhibitor in the treatment of endothelial dysfunction and/or in a low-dose proteasome inhibitor therapy

(75) Inventors: Verena Stangl, Berlin (DE); Karl Stangl, Berlin (DE); Mario Lorenz, Berlin (DE)

Correspondence Address: ROTHWELL, FIGG, ERNST & MANBECK, P.C., 1425 K STREET, N.W., SUITE 800, WASHINGTON, DC, 20005, US

(73) Assignee: Charite'-Universitaetsmedizin Berlin, Berlin (DE)

(21) Appl. No.: 10/522,706

(22) PCT Filed: Jul. 31, 2003
(86) PCT No.: PCT/EP03/08495

§ 371 Date: Jan. 28, 2005

(30) Foreign Application Priority Data
Jul. 31, 2002 (EP) 02017234.2

(51) Int. Cl. A61K 38/06; A61K 38/05; A61K 38/04; A61K 31/69

(52) U.S. Cl. 514/18; 514/19; 514/64

(57) ABSTRACT

The present invention relates to the use of a proteasome inhibitor for the manufacture of a medicament for the prevention, onset therapy, acute therapy and/or regression of diseases associated with endothelial dysfunction. The present invention also relates to the use of a proteasome inhibitor as a low-dose treatment.

The present invention refers to the use of a proteasome inhibitor for the manufacture of a medicament for the prevention, onset therapy, acute therapy and/or regression of diseases associated with endothelial dysfunction.

The present invention also refers to the use of a proteasome inhibitor for the manufacture of a medicament for the prevention, onset therapy, acute therapy and/or regression of diseases using said proteasome inhibitor for a low-dose treatment.

Nitric oxide (NO) is an important anti-atherogenic molecule, and NO-based interventions represent a powerful approach against restenosis.

Endothelial nitric oxide synthase (eNOS) is a key regulator of vascular wall homeostasis. Its product, nitric oxide (NO), mediates shear-stress induced endothelial-dependent vasodilation and exerts pronounced anti-atherogenic effects. Reduced NO generation and/or bioavailability has been implicated in the pathophysiology of several disease states such as coronary artery disease, hypertension, diabetes, and heart failure (Kojda G, Harrison D. Interactions between NO and reactive oxygen species: pathophysiological importance in atherosclerosis, hypertension, diabetes and heart failure. Cardiovasc Res. 1999; 43:562-571; Oemar B S, et al. Reduced endothelial nitric oxide synthase expression and production in human atherosclerosis. Circulation. 1998; 97:2494-2498, Zeiher A M, et al. Endothelium-mediated coronary blood flow modulation in humans. Effects of age, atherosclerosis, hypercholesterolemia, and hypertension. J Clin Invest. 1993; 92:652-662, Busse R, Fleming I. Endothelial dysfunction in atherosclerosis. J Vase Res. 1996; 33:181-194, Treasure C B, et al. Endothelium-dependent dilation of the coronary microvasculature is impaired in dilated cardiomyopathy. Circulation. 1990; 81:772-779).

Regulation of eNOS occurs at the transcriptional, post-transcriptional, and post-translational level. Whereas increases in intracellular calcium and phosphorylation induce rapid transient elevation of eNOS activity—allowing fast response to changing environmental conditions (Dimmeler S, et al. Activation of nitric oxide synthase in endothelial cells by Akt-dependent phosphorylation. Nature. 1999; 399:601-605, Dimmeler S, Dembach E, Zeiher M. Phosphorylation of the endothelial nitric oxide synthase at Ser-1177 is required for VEGF-induced endothelial cell migration. FEBS Letters. 2000; 477:258-262)—sustained alterations are primarily due to changes in the expression level of eNOS protein (Wu K K. Regulation of endothelial nitric oxide synthase activity and gene expression. Ann N Y Acad Sci. 2002; 962:122-130). A large number of stimuli are known to increase the expression of eNOS including growth factors such as VEGF (Hood J D, Meininger C J, Ziche M, et al. VEGF upregulates ecNOS message, protein, and NO production in human endothelial cells. Am J Physiol. 1998; 274: H1054-H1058), EGF and bFGF (Zheng J, Bird I M, Melsaether A N, et al. Activation of the mitogen-activated protein kinase cascade is necessary but not sufficient for basic fibroblast growth factor- and epidermal growth factor-stimulated expression of endothelial nitric oxide synthase in ovine fetoplacental artery endothelial cells. Endocrinology. 1999; 140:1399-1407), as well as TGF-β(Inoue N, Venema R C, Sayegh H S, et al. Molecular regulation of the bovine endothelial nitric oxide synthase by transforming growth factor-beta 1. Arterioscler Thromb Vase Biol. 1995; 15:1255-1261); hormones such as insulin (Kuboki K, Jiang Z Y, Takahara N, et al. Regulation of endothelial constitutive nitric oxide synthase gene expression in endothelial cells and in vivo. A specific vascular action of insulin. Circulation. 2000; 101:676-681) and estrogen (Kleinert H, et al. Estrogens increase transcription of the human endothelial NO synthase gene. Analysis of the transcription factors involved. Hypertension. 1998; 31:582-588); HMG-coenzyme A reductase inhibitors (Laufs U, et al. Upregulation of the endothelial nitric oxide synthase by HMG CoA reductase inhibitors. Circulation. 1998; 97:1129-1135); and mechanical forces such as shear stress (Ziegler T, et al. Nitric oxide synthase expression in endothelial cells exposed to mechanical forces. Hypertension. 1998; 32:351-355).

The ubiquitin-proteasome system represents the major pathway for intracellular protein degradation in eukaryotic cells (Rock K L, et al. Inhibitors of the proteasome block degradation of most cell proteins and the generation of peptides presented on MHC class I molecules. Cell. 1994; 78:761-771). The 26S proteasome consists of a proteolytic core complex, the 20S proteasome, and two 19S regulatory complexes (Coux O, Tanaka K, Goldberg A L. Structure, and functions of the 20S, 26S proteasomes. Annu Rev Biochem. 1996; 65:801-847, Hershko A, Ciechanover A, Varshavsky A. The ubiquitin system. Nat Med. 2000; 10:1073-1082). Before degradation, the substrates are labelled by conjugation with multi-ubiquitin chains. Rapid degradation of many rate-limiting enzymes and transcription factors is catalyzed by the proteasome.

Activation of eNOS and enhancement of vasorelaxation by proteasome inhibition represent potentially important cardiovascular protective effects. The ubiquitin-proteasome pathway may accordingly represent a novel drug target to improve endothelial function.

Musial A and Eissa T (Inducible nitric-oxide synthase is regulated by the proteasome degradation pathway. J Biol Chem. 2002; 276:24268-242273) have identified the proteasome as the primary degradation pathway for inducible NOS.

WO 98/35691 is directed to treating ischemia and reperfusion injury after ischemia by administering proteasome inhibitors, ubiquitin pathway inhibitors, agents that interfere with the activation of NF-κB via the ubiquitin proteasome pathway, or mixtures thereof. According to specific embodiments of this application, the proteasome inhibitor is selected from the group of a peptidyl aldehyde, a peptidyl boronic acid or peptidyl boronic ester, a lactacystin analog, N-(2-pyrazine) carbonyl-L-phenylalanine-L-leucine boronic acid and 7-n-propyl-clasto-lactacystin-β-lactone.

WO 02/05810 describes methods of modulating endothelial NOS (eNOS) expression, e.g., insulin stimulated eNOS expression, by modulating PKCβ. The methods described are useful in the treatment of insulin-related disorders, e.g., hypertension, diabetes, atherosclerosis, ischemia, or insulin resistance.

Luss et al. (Luss H, Schmitz W, Neumann J. A proteasome inhibitor confers cardioprotection. Cardiovasc Res. 2002; 54:140-51) describe for several cell types that proteasome inhibitors like carbobenzoxyl-leucinyl-leucinyl-leucinal (MG132) induce the 72 kDa heat shock protein (Hsp72) and exert cell protective effects. These authors investigated the effects of MG132 in cultured neonatal rat cardiomyocytes and it was found that MG132 time- and concentration-dependently induced Hsp72 and Hsp32 at mRNA and protein levels. Although Hsp60 mRNA was induced, Hsp60 protein levels were not altered. MG132 (1 μM) prolonged the spontaneous beating time of cardiomyocytes at 46° C. from 5+/−2 min (control hyperthermia) to 28+/−5 min (P<0.05, n=4). Thus, inhibition of the proteasome function by MG132 protects cardiomyocytes against hyperthermic or oxidative injury and might be a novel cardioprotective modality.

Finally, Stangl et al. (Stangl K, Gunther C, Frank T, Lorenz M, Meiners S, Ropke T, Stelter L, Moobed M, Baumann G, Kloetzel P M, Stangl V. Biochem Biophys Res Commun 2002 Mar 1; 291(3):542-9 “Inhibition of the ubiquitin-proteasome pathway induces differential heat-shock protein response in cardiomyocytes and renders early cardiac protection.”) disclose the effects of proteasome inhibition on heat-shock protein (HSP) expression in cardiomyocytes with MG132 (0.1-10 μM) and MG262. In these experiments, concentrations of less than 1 μM did not exhibit protective effects when applied to the cardiomyocytes.

Taken together, the above disclosures describe several indications that can be treated using proteasome inhibitors at elevated concentration levels that might be difficult to achieve in the human body. In addition, further therapeutical approaches based on proteasome inhibitors are lacking.

Therefore, it is an object of the present invention to provide for novel approaches in order to improve therapeutical approaches known in the art using proteasome inhibitors. It is another object of the present invention, to provide for novel approaches in order to improve endothelial function by proteasome inhibition. These novel approaches should provide for a long-term effective therapy based on as low as possible amounts of medication.

The object of the present invention is solved by the use of at least one proteasome inhibitor for the manufacture of a medicament for the prevention, onset therapy, acute therapy and/or regression of diseases associated with endothelial dysfunction.

The object of the present invention is further solved by the use of at least one proteasome inhibitor for the manufacture of a medicament for the prevention, onset therapy, acute therapy and/or regression of a disease selected from a group comprising a disease associated with endothelial disfunction, wherein the proteasome inhibitor dose provided to a patient in need is of low-dose, i.e. in the nmolar range.

The object of the present invention is also solved by a method for the prevention, onset therapy, acute therapy and/or regression of diseases associated with endothelial dysfunction, comprising applying to the patient in need a therapeutically effective amount of at least one proteasome inhibitor.

The object of the present invention is also solved by a method for the prevention, onset therapy, acute therapy and/or regression of a disease selected from a group comprising a disease associated with endothelial dysfunction, comprising applying to the patient in need a proteasome inhibitor, wherein the proteasome inhibitor dose provided is of low dose, i.e. in the nmolar range.

In one embodiment of the present invention, the diseases associated with endothelial dysfunction are non-insulin related diseases.

In another embodiment of the present invention, endothelial dysfunction is associated with atherosclerosis, in particular coronary sclerosis and coronary artery disease.

In another embodiment of the present invention, endothelial dysfunction is associated with heart failure.

In just another embodiment of the present invention, endothelial dysfunction is associated with diseases resulting from ischemia and/or reperfusion injury of organs and/or of parts of the body selected from the group comprising heart, brain, peripheral limb, kidney, liver, spleen and lung, and/or wherein the endothelial dysfunction is associated with diseases selected from a group comprising infarctions such as myocardial infarction and critical limb ischemia, and/or wherein the endothelial dysfunction is associated with diseases selected from the group comprising ischemic diseases such as peripheral arterial occlusive disease, e.g. critical leg ischemia, myocardial infarction and ischemic diseases of organs, e.g. of the kidney, spleen, brain and lung.

In a preferred embodiment of the present invention, the proteasome inhibitor is selected from a group comprising:
a) naturally occurring proteasome inhibitors comprising:
   peptide derivatives which have a C-terminal expoxy ketone structure, β-lactone-derivatives, aclacinomycin A, lactacystin, clastolactacystein;
b) synthetic proteasome inhibitors comprising:
   modified peptide aldehydes such as N-carbobenzoxy-L-leucinyl-L-leucinyl-L-leucinal (also referred to as MG132 or zLLL), or the boronic acid derivative of MG232, N-carbobenzoxy-Leu-Nva-H (also referred to as MG115), N-acetyl-L-leucinyl-L-leucinyl-L-norleucinal (also referred to as LLnL), N-carbobenzoxy-Ile-Glu(OBut)-Ala-Leu-H (also referred to as PS1);
c) peptides comprising:
   an α,β-epoxyketone-structure, vinyl-sulfones such as, carbobenzoxy-L-leucinyl-L-leucinyl-L-leucin-vinyl-sulfone or, 4-hydroxy-5-iodo-3-nitrophenylacetyl-L-leucinyl-L-leucinyi-L-leucin-vinyl-sulfone (NLVS);
d) Glyoxal- or boric acid residues such as: pyrazyl-CONH(CHPhe)CONH(CHisobutyl)B(OH)₂ and dipeptidyl-boric-acid derivatives;
e) Pinacol-esters such as: benzyloxycarbonyl(Cbz)-Leu-leuboro-Leu-pinacol-ester.

In a preferred embodiment of the present invention, the proteasome inhibitor is selected from a group comprising PS-314 as a peptidyl-boric-acid derivative which is N-pyrazinecarbonyl-L-phenylalanine-L-leucine-boric acid (C₁₉H₂₅BN₄O₄); PS-519 as a β-lactone- and a lactacys-tin-derivative which is 1R-[1S,4R,5S]-1-(1-hydroxy-2-methylpropyl)-4-propyl-6-oxa-2-azabicyclo[3.2.0]heptane-3,7-dione (C₁₂H₁₉NO₄); PS-273 (morpholino-CONH—(CH-naphthyl)-CONH—(CH-isobutyl)-B(OH)₂) and its enantiomer; PS-293; PS-296 (8-quinolyl-sulfonyl-CONH—(CH-naphthyl)-CONH(—CH-isobutyl)-B(OH)₂); PS-303 (NH₂(CH-naphthyl)-CONH—(CH-isobutyl)-B(OH)₂; PS-321 as (morpholino-CONH—(CH-naphthyl)-CONH—(CH-phenylalanine)-B(OH)₂); PS-334 (CH₃—NH—(CH-naphthyl-CONH—(CH-isobutyl)-B(OH)₂); PS-325 (2-quinol-CONH—(CH-homo-phenylalanine)-CONH—(CH-isobutyl)-B(OH)₂; PS-352 (phenyalanine-CH₂—CH₂—CONH—(CH-isobutyl)-1-B(OH)₂; PS-383 (pyridyl-CONH—(CHₚF-phenylalanine)-CONH—(CH-isobutyl)-B(OH)₂); PS-341; and PS-1 Z-Ile-Glu(OtBu)-Ala-Leu-CHO; PS-2 [Benzyloxycarbonyl)-Leu-Leu-phenylalaninal or Z-LLF-CHO or Z-Leu-Leu-Phe-CHO PS-1; PS-519 as a β-lactone- and a lactacystin-derivative which is 1R-[1S,4R,5S]-1-(1-hydroxy-2-methylpropyl)-4-propyl-6-oxa-2-azabicyclo[3.2.0]heptane-3,7-dione (C₁₂H ₁₉NO₄); epoxomicin (C₂₈H₈₆N₄O₇) and eponemycin (C₂₀H₃₆N₂O₅).

In a preferred embodiment, the proteasome inhibitor is selected from a group comprising a peptide aldehyde, a peptide boronate, a peptide vinylsulfone, a peptide epoxyketone, a lactacystin, a peptide α-ketonaldehyde, an α-ketoamide, an indanonpeptide, a polyalkylenalde-hyde, a polyphenol such as cathechin-3-gallate.

In just another preferred embodiment, the proteasome inhibitor is selected from a group comprising Z-Leu-Leu-Leu-al (MG132), Z-Ile-Glu(OtBu)-Ala-Leu-al (PS-1), CEP1612, pyrazyl-carbonyl-Phe-Leu-boronate (PS-341), dansyl-Phe-Leu-boronate (DFLB), morpholino-naphthylalanine-Leu-boronate (MG273), NIP-Leu₃-vinylsulfone (NLVS), Tyr-Leu₃-VS, NIP-Leu-Leu-Asn-VS, Ada-Tyr-Ahx₃-Leu₃-VS, Ada-Lys(bio)-Ahx₃-Leu₃-VS, Ac(Me)-Ile-Ile-Thr-Leu-EX (epoxomicin), dihydroeponemycin, lactacystin, clasto-lactacystin-β-lactone (omuralide), PS-519, Ac-Leu-Leu-Nle-al (ALLN), 3,4-dichloro-isocoumarin (DCl), 4-(2-aminoethyl)-benzenesulfonylfluoride (Pefablock SC), TMC-95-A, gliotoxin, (−)epigallocatechin-3-gallate (EGCG), ritonavir, lovastatin, aclacinomicin A (aclarubicin), cyclosporin, wherein Z represents benzyl oxycarbonyl, all represents aldehyde, VS represents vinylsulfone, NIP represents 3-nitro-4-hydroxy-5-iodophenylacetate, and bio represents biotin.

Preferentially, the proteasome inhibitor is MG132.

In another preferred embodiment, the proteasome inhibitor interferes with gene expression of at least one component of the proteasome complex.

In a preferred embodiment, the proteasome inhibitor interfering with proteasomal gene expression is selected from a group comprising antisense RNA, double stranded RNA and oligonucleotides hybridising with a DNA sequence encoding at least one component of the proteasome complex.

In another preferred embodiment, the proteasome inhibitor interfering with proteasomal gene expression is selected from a group comprising a knock out construct.

As used herein, the proteasome inhibitor used for the treatment according to the present invention can be selected from a broad variety of available proteasome inhibitors. Such inhibitors generally include all substances that interfere with the formation and/or action of the proteasome complex. For a selection of proteasome inhibitors see Kisselev A F and Goldberg A L (Chemistry & Biology. 2001; 8: 739-758), which proteasome inhibitors are hereby incorporated by reference. For commercially available proteasome inhibitors see also the catalogue listing of A.G. Scientific, Inc. (http://www.proteasomes.com), which proteasome inhibitors are hereby incorporated by reference.

The terms “prevention”, “onset therapy”, “acute therapy” and “regression” in the context of the present invention are used in accordance with their meaning commonly known by a skilled person.

As used herein, the term “endothelial dysfunction” is used in accordance with its meaning commonly known to someone skilled in the art. In particular, it is meant to designate any functional, structural, and/or physiological abnormality of the endothelium.

According to one aspect of the invention, the proteasome inhibitor is provided to a patient in need in a very low amount. Surprisingly, a single low-dose MG132 treatment (100 nmol/L) induced long-term effects in CPAE cells (a bovine pulmonary artery endothelial cell line, ATCC, #CCL-209), with increases of eNOS protein and enzyme activity up to 10 days. Adapted to a patient in need, the proteasome inhibitor MG132 would be administered in an amount far below the amounts intended for administration today. This low amount would comprise an amount which finally results in a therapeutically effective concentration of between 1-100 nmol/L, i.e. in the nmolar range. Of course, the individual amounts to be applied will differ based on several factors that are associated with the specific medication, such as, the proteasome inhibitor used, the weight of the patient, the specific disease to be treated, the severity and so on. These factors are all known to the skilled person and can therefore easily changed and distinguished in order to fit to the specific therapeutical situation and requirements.

Finally, provided is a method for the prevention, onset therapy, acute therapy and/or regression of diseases associated with endothelial dysfunction, comprising applying to the patient in need a therapeutically effective amount of least one proteasome inhibitor.

The inventors have recently shown that inhibition of the ubiquitin-proteasome system prevents and reduces neointima formation and restenosis in rat carotid arteries, whereupon the inventors subsequently investigated a possible link between the proteasome and eNOS. Since NO has been shown to have anti-atherogenic properties and NO-based interventions appear to represent a potentially powerful approach toward prevention of restenosis (Janero D R and Ewing J F. Nitric oxide and postangioplasty restenosis: pathological correlates and therapeutic potential. Free Radical Biol Med. 2000; 29:1199-1221), one object was to investigate whether there is a link between the ubiquitin-proteasome pathway and eNOS. It has not been known until now whether the proteasome regulates eNOS.

Endothelial nitric oxide synthase (eNOS) is a key regulator of vascular wall homeostasis. Here the inventors show that the 26S proteasome regulated eNOS expression in endothelial cells. Blocking the proteasome with very low, non-toxic, single inhibitor doses resulted in increased eNOS expression. This upregulation enhanced eNOS activity and led to augmented NO-dependent vasorelaxation in pretreated rat aortas. Proteasome-induced eNOS upregulation represented a long-term effect that lasts up to ten days.

The findings of the inventors that the ubiquitin-proteasome system is an important regulator of the eNOS enzyme elucidate another aspect of this degradative system entailing potential therapeutic implications. Whereas inhibitors of the proteasome system have been shown to have impressive potential for treatment of cancer and inflammation by virtue of their anti-proliferative, anti-inflammatory, and pro-apoptotic effects, accumulating evidence now indicates the possibility of extending the potential of these inhibitors to integrative organ protection. Inhibitors that block proteasome function have accordingly been shown to induce heat-shock response in various cell types, e.g., HepG2, canine kidney, and human intestinal epithelial cells. In addition, the inventors and others have recently shown that proteasome inhibition induces differential heat-shock response in cardiomyocytes, accompanied by enhanced cell survival and improved functional recovery after various forms of stress.

In this study the inventors describe how inhibition of the ubiquitin-proteasome system has important effects on endothelial cells, in the increase of eNOS expression and activity, with consequent improvement of endothelial function. NO mediates endothelial-dependent vasodilation and exhibits potent atheroprotective properties. Reduction in eNOS activity or NO bioavailability is one of the earliest manifestations in atherosclerosis, and approaches that may prevent or antagonize these effects by activating eNOS may represent a beneficial therapeutic tool.

The present study indicates, without wishing to be bound by any theory, that proteasome inhibition upregulates eNOS mRNA. Upregulation of eNOS by proteasome inhibition occurs after 16 to 24 hours, with results in the order of magnitude as described for strong physiological stimuli such as estrogen and for potent drugs such as statins. With regard to the fact that even a 30% change in eNOS activity can induce corresponding increases in blood flow 31, the rise in eNOS activity by 200 to 300% observed after proteasome inhibition is significant. The inventors accordingly observed a significant increase in endothelial-dependent vasorelaxation in MG132 pretreated rat vascular rings.

In order to avoid cytotoxicity, the inventors determined the lowest inhibitor doses capable of enhancing eNOS expression. Surprisingly, in the endothelial cell cultures used, eNOS. was already upregulated by very low inhibitor doses. Accumulation of poly-ubiquitinylated proteins occurring at these low concentrations indicates that this eNOS upregulation was indeed attributable to inhibition of the proteasome. In targeting of a common, basic regulatory system such as the proteasome, the putative mechanism by which such low inhibitor doses exert specific cardiovascular protective effects remains unclear. One may speculate that proteasome inhibitors in such low doses act only by partial inhibition, in a subunit-specific manner, and/or by differentially blocking subtypes of the 20S proteasome.

Interestingly, a single, low-dose of MG132 upregulates eNOS expression up to 10 days, although the effect of proteasome inhibition is observed only until day 3. It may be further speculated that endothelial cells experience a phenotype shift, and gain in turn the ability of higher NO production. The inventors are not aware of another substance for which similar long-term upregulation of eNOS has been demonstrated.

Most important, the inventors have shown that proteasome inhibitors are capable to reverse and/or alleviate the effects of endothelium damaging agents.

In summary, the inventors have identified the ubiquitin-proteasome pathway as an important regulator of eNOS. By significantly increasing eNOS expression and activity for several days, proteasome inhibitors enhance the availability of nitric oxide in endothelial cells and increase endothelium-dependent vasodilatory capacity in rat aortic rings. Targeting the proteasome system therefore provides a novel therapeutic concept for amelioration of endothelial functions, in terms of improved vasodilation as well as by amplification or restoration of anti-atherogenic properties.

The invention shall now be further explained by the following examples, without being limited thereto, with respect to the accompanying drawings in which:

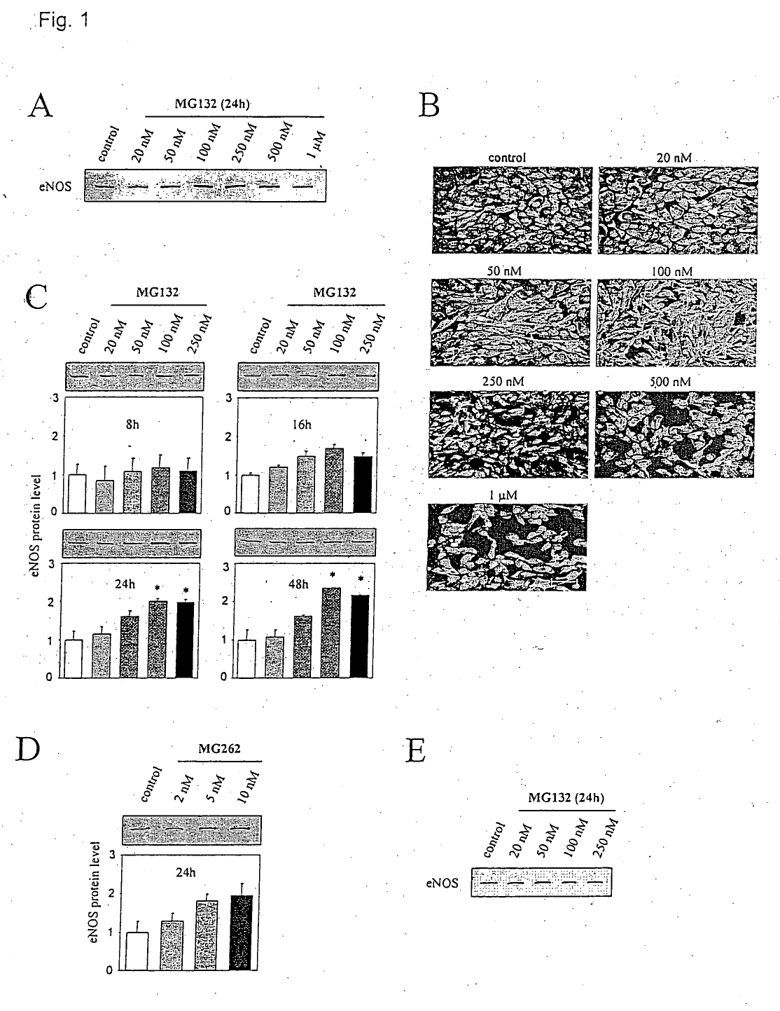

FIG. 1 shows the effects of proteasome inhibition on eNOS protein expression and cell morphology in CPAE cells. A, Representative Western blot, showing gradual upregulation in eNOS expression after incubation of CPAE cells with MG132 in concentrations up to 250 nmol/L, and a decrease in eNOS protein levels at higher doses of the proteasome inhibitor. B, Representative microscopic scan of CPAE cells, double-stained with phalloidin and DAPI after incubation (24 hours), with the indicated doses of MG132 illustrating dose-dependent morphological changes of the cells. The bar represents 100 μm. C, Representative Western blot and densitometric analysis (mean±SEM) of time- and dose-dependent effects of MG132 compared to control (solvent). There was significant upregulation of eNOS beginning 24 hours after incubation of the CPAE cells with MG132 at 50 nmol/L. n=3 separate experiments. *P<0.05 versus control. D, Western blot, showing that MG262 also upregulates eNOS in CPAE cells. E, In HUVECs Western blots disclose dose-dependent increase in eNOS after incubation of the cells with MG132,

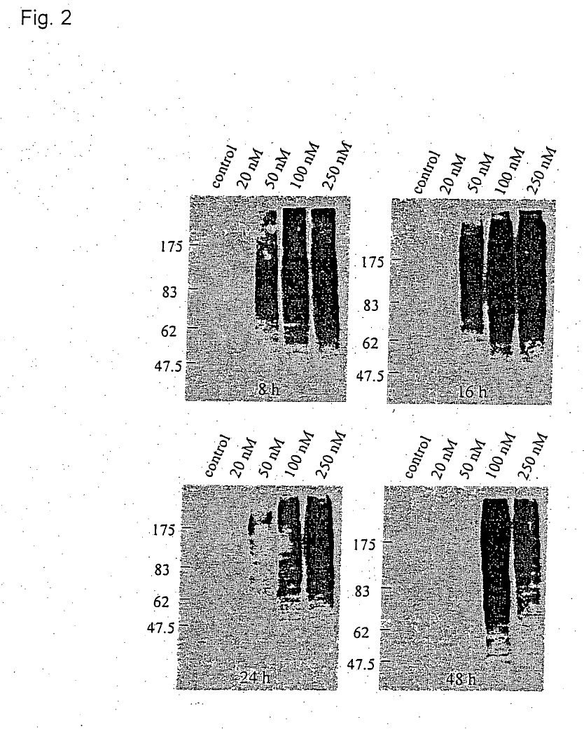

FIG. 2 shows representative Western blot (anti-ubiquitin antibody) showing the efficacy of proteasome inhibition by very low concentrations of MG132 in CPAE cells: beginning with 50 nmol/L, MG132 led to accumulation of poly-ubiquitinylated proteins. On the left, molecular weight (kD) markers.

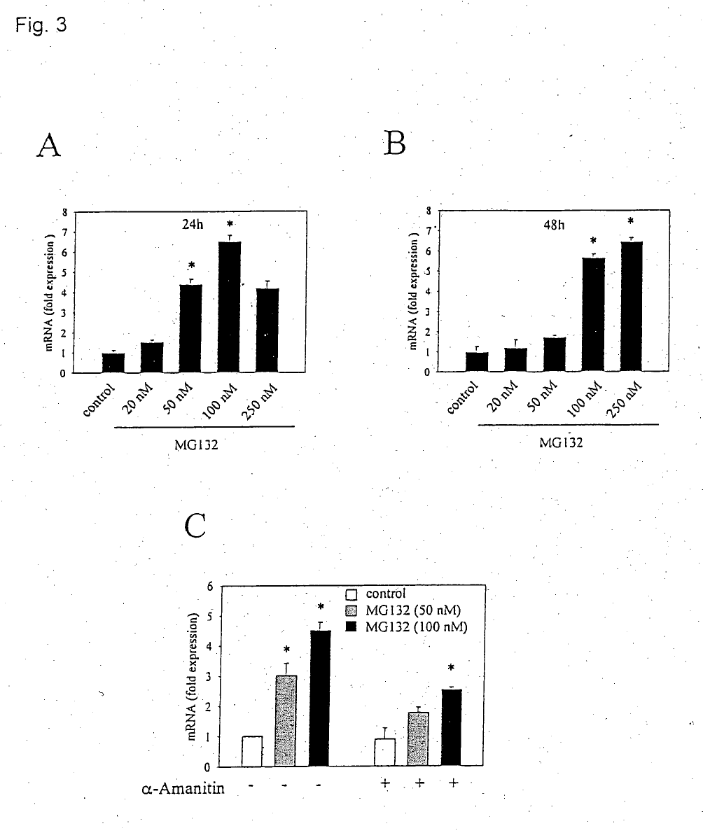

FIG. 3 shows the effects of proteasome inhibition on eNOS mRNA levels using realtime RT-PCR. CPAE cells were treated for 24 (A) and 48 (B) hours with solvent (control) or MG132 (20-250 nmol/L). Values are mean±SEM from 3 separate experiments. C, Pretreatment (1 hour) of CPAE cells with the transcription inhibitor α-amanitin (2.5 μg/L) before addition of the proteasome inhibitor reduced overall eNOS mRNA, without eliminating an MG132-induced increase in eNOS mRNA. Values are mean±SEM obtained from 3 separate experiments. *P<0.05 versus control,

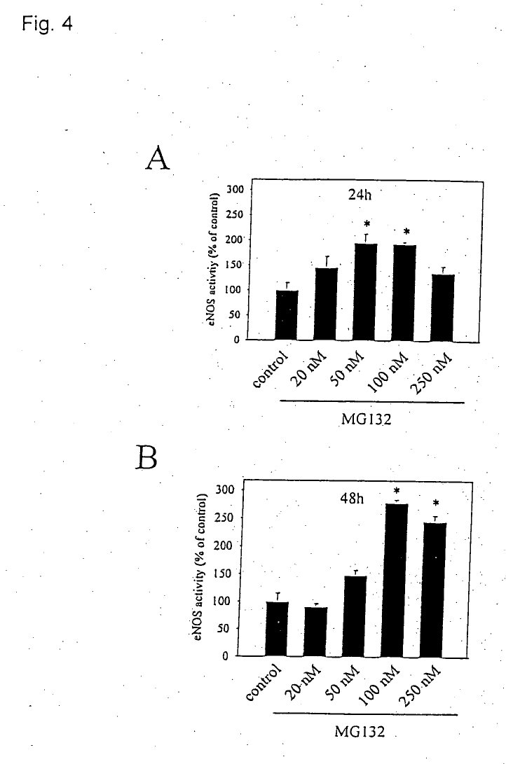

FIG. 4 shows that the inhibition of the proteasome increases eNOS activity in CPAE cells. L-[³H]arginine conversion to L-[³H]citrulline measured under control conditions (solvent) or in the presence of increasing concentrations of MG132 at 24 hours (A) and 48 hours (B). Values are mean±SEM, n=3. *P<0.05 versus control.

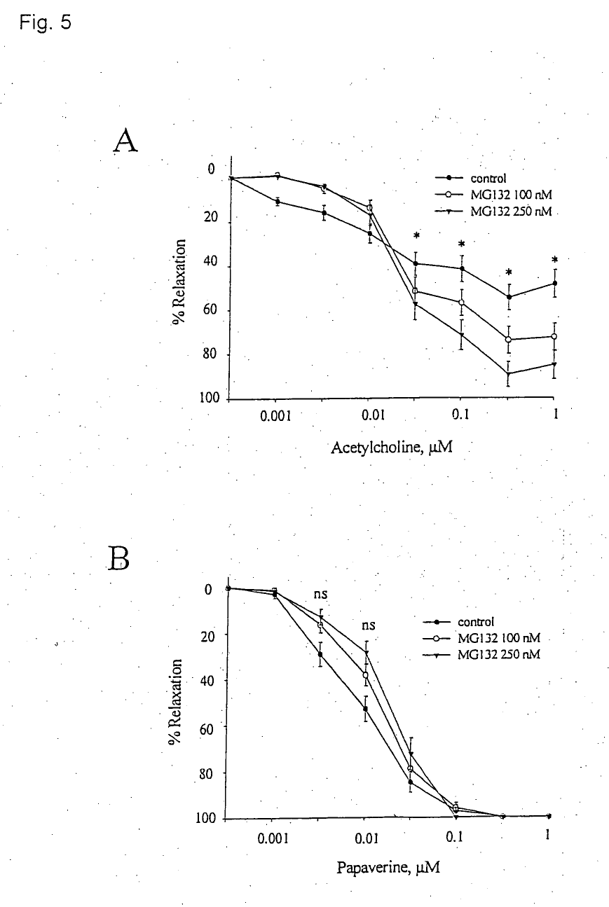

FIG. 5 shows the effects of pretreatment with MG132 (100 and 250 nmol/L, 48 hours) on vasoreactivity in rat aortic rings. Endothelial-dependent vasorelaxation was tested with acetylcholine (A), and endothelial-independent vasodilation was investigated with papaverine (B). The drugs were added with increasing concentrations to obtain cumulative concentration-response curves. Graphs show relaxation expressed as percentage of maximal phenylephrine-induced vasoconstriction. There was significant increase in endothelial-dependent vasorelaxation in MG132-treated aortic rings, whereas endothelial-independent vasodilation remained unaffected. Data are expressed as mean±SEM (n=6-8 aortas). *P<0.05 versus vehicle-treated rings. ns, not significant.

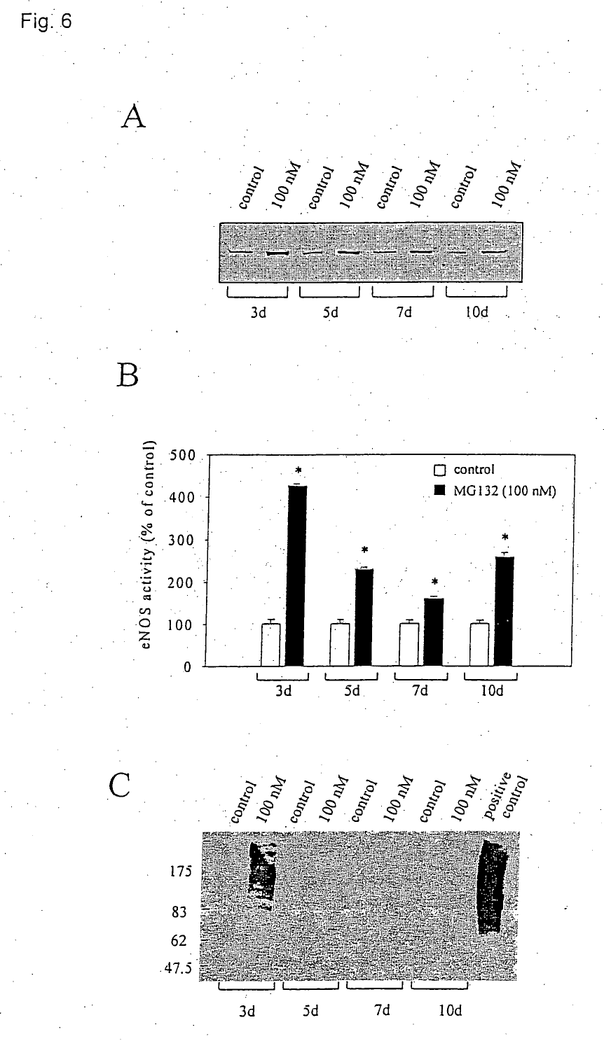

FIG. 6 shows the long-term effects of a single dose of MG132 in CPAE cells. Representative Western blot showing upregulation of eNOS protein (A) and, correspondingly, a significant increase in eNOS activity (B), up to 10 days following proteasome inhibition. Values are mean±SEM, n=3. C, Representative Western blot demonstrating that accumulation of poly-ubiquitinylated proteins was detectable no longer than 3 days. Positive control indicates MG132 treatment for 24 hours. *P<0.05 versus control.

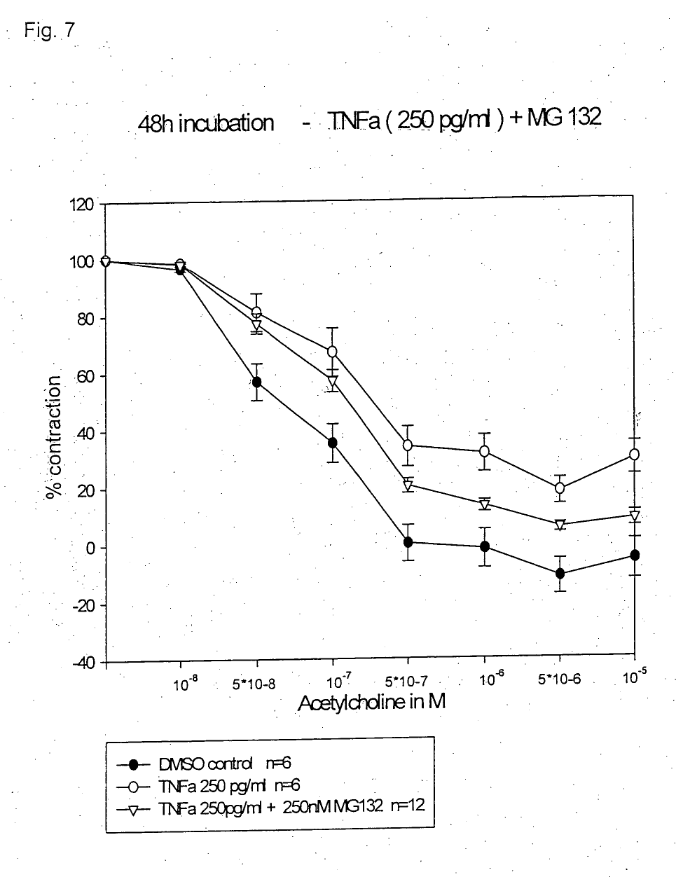

FIG. 7 shows the protective effect of proteasome inhibitors (here MG132) on vasoreactivity in rat aortic rings. Rat aortic rings were precontracted by means of phenylephrine. Endothelial-dependent vasorelaxation was tested with acetylcholine. The aortic rings were preincubated with either DMSO (control) for 48 hours, TNF-α for 48 hours, TNF-α having a damaging effect on vasoreactivity in aortic rings, or TNF-α together with MG132 for 48 hours. The figure shows three experiments performed on rat aortic rings which experiments were performed either with a control (DMSO as solvent), or with TNF-α (250 pg/ml) or with TNF-α (250 pg/ml)+MG132 (250 nM). Acetylcholine was added in increasing concentrations to obtain cumulative concentration-response curves. The graph shows the contraction expressed as a percentage of the maximal phenylephrine-induced vasoconstriction, with 100% contraction meaning the initial state induced by phenylephrine.

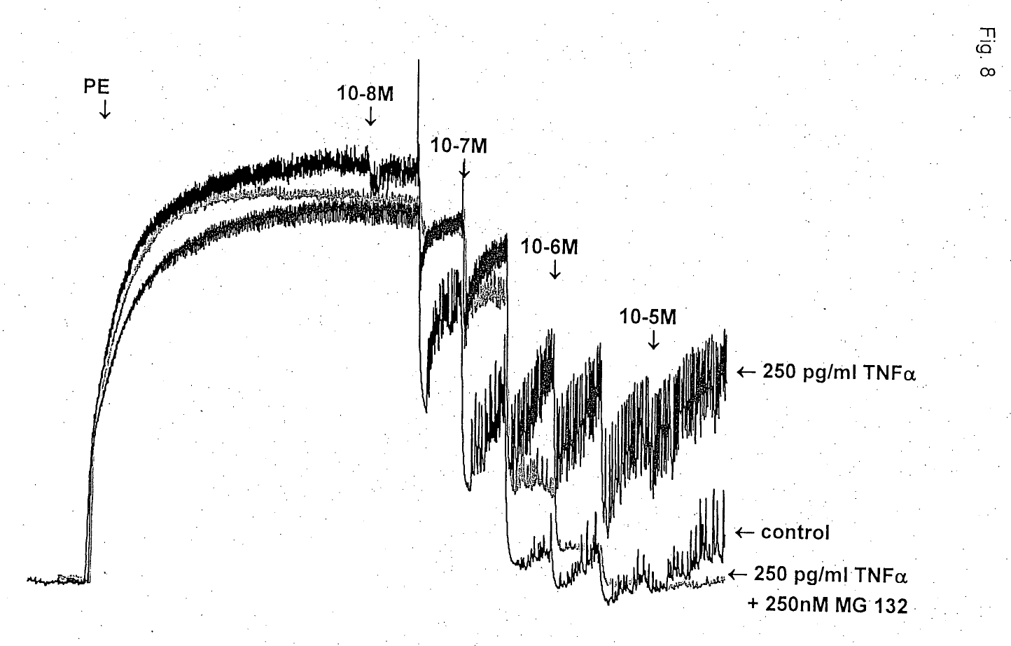

FIG. 8 shows the original trace of single experiments a series of which were performed in FIG. 7. “PE” denotes the initial application of phenylephrine; the subsequent arrows indicate an application of the corresponding concentrations of acetylcholine (i.e. 10⁸ M; 10⁷ M, 10⁶ M, 10⁵ M). Three traces are shown, one of which is control (DMSO only), the other ones being 250 pg/ml TNF-α with and without 250 nM MG132.

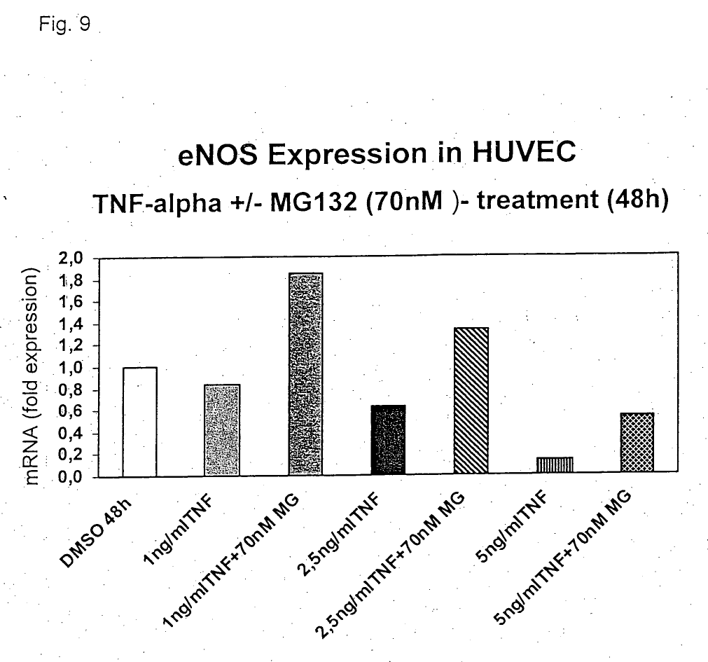

FIG. 9 shows the effects of a 48 hours treatment of DMSO alone, TNF-α (1 ng/ml, 2.5 ng/ml and 5 ng/ml) with or without 70 nM MG (=MG132) on the expression of eNOS mRNA in HUVECs.

### MATERIALS AND METHODS

1. Materials and Statistical Analysis

Proteasome inhibitors MG132, MG262, and the non-proteasomal cathepsin inhibitor ALLM (N-acetyl-leucyl-leucyl-methioninal) were obtained from CalBiochem (San Diego, Calif.). The transcription inhibitor α-amanitin was purchased from Roche Diagnostics (Mannheim, Germany); L-arginine from Amersham (Freiburg, Germany); and Dowex AG50WX-8, phenylephrine, acetylcholine, and papaverine, from Sigma Chemical (Deisenhofen, Germany). Anti-eNOS monoclonal antibody was purchased from BD Transduction Laboratories (Heidelberg, Germany), and anti-ubiquitin antibody from DAKO (Hamburg, Germany). Secondary anti-mouse antibody was obtained from Santa Cruz Biotechnology (Santa Cruz, Calif.), and anti-rabbit antibody from Dianova (Hamburg, Germany).

All values are expressed as mean±SEM compared to controls. Band intensities were analysed by densitometry. Vasorelaxation is expressed as percentage of precontraction with phenylephrine. Statistical analysis was performed by use of ANOVA, Mann-Whitney, or Student's Test where appropriate. A level of P<0.05 was considered significant in all statistical tests.

2. Methods

Method 1: Cell Culture and Treatments

The bovine pulmonary artery endothelial cell line (CPAE cells) was purchased from the American Type Culture Collection (ATCC, # CCL-209) and was cultured in MEM supplemented with 5% FCS, 1.5 g/L sodium bicarbonate, 0.11 g/L sodium pyruvate, 100 U/mL penicillin, and 100 μg/ml streptomycin. Human umbilical vein endothelial cells (HUVECs) were isolated and cultured as described previously (Stangl et al. Homocysteine inhibits TNF-α-induced endothelial adhesion molecule expression and monocyte adhesion via nuclear factor-κB dependent pathwas. Biochem Biophys Commun. 2001; 280:1093-1100). Purity of HU-VECs was assessed by von Willebrand factor staining. For experiments, cells were seeded onto 6-mm diameter dishes and were treated with either proteasome inhibitors MG132, MG262, with the non-proteasomal cathepsin inhibitor ALLM, or with solvent (DMSO) for the times and concentrations indicated. In some experiments, the transcription inhibitor α-amanitin (2.5 μg/mL) was pre-incubated one hour before addition of proteasome inhibitor. Cell viability was assessed by trypan blue exclusion. For HUVECs, the inventors used only second- to third-passage cells for all experiments. For phalloidin-DAPI double staining, cells were fixed with 4% paraformaldehyde, permeabilized with 1% Triton, and blocked with 1% FCS. The inventors took cell photos with a Zeiss camera and analysed them with Axiovert 3.0 software.

For eNOS expression in HUVECs, cells were treated with DMSO (control), TNF-α (1, 2.5 and 5 ng/ml) and TNF-α (same concentrations) in the presence of 70 nM MG132, for 48 hours, whereupon eNOS mRNA levels were measured (see Method 3).

Method 2: Western Blot Analysis

After treatment, cells were washed twice with PBS and lysed in extraction buffer containing in mmol/L: Tris/HCl 50 (pH 7.4), KCl 154, glucose 5, EDTA 0.5, PMSF 1, DTT 2, and 1% Triton X-100. Total protein (50 μg per lane) was subjected to SDS-PAGE and membranes were probed with anti-eNOS monoclonal antibody (1:2500) or with anti-ubiquitin antibody (1:1000). Membranes were incubated with secondary anti-mouse antibody (1:2500) conjugated to alkaline phosphatase for eNOS or with anti-rabbit antibody conjugated to horseradish peroxidase (1:10000) for ubiquitin. Bands were visualized by using BCIP and Nitro Blue Tetrazolium (Sigma) for eNOS, and by employing the ECL detection system (Amersham) for ubiquitin.

Method 3: Real Time-PCR (RT-PCR)

After treatment, cells were lysed in Trizol reagent (Gibco Life Technologies, Karlsruhe, Germany) and 800 ng of total RNA were reversed transcribed with random hexamers. Primer sequences were synthesized by TIB Molbiol (Berlin, Germany), and TaqMan probes by Eurogentec (Seraing, Belgium). Table 1 summarizes the sequences of primers and probes used in this study. mRNA expression was standardized to the HPRT (hypoxanthine phosphoribosyl transferase) gene as a housekeeping gene, the transcription level of which were not influenced under our experimental conditions. PCR amplification was carried out in 25 μL TaqMan Universal PCR Master Mix (Perkin Elmer Applied Biosystems, Foster City, Calif.) containing either 0.3 or 0.9/μmol/L primer, 0.2/μmol/L TaqMan probe, and 1 μl of the reverse transcription reaction in a 5700 Sequence Detection System (Perkin Elmer Applied Biosystems). Thermal cycling conditions comprised an initial denaturation step at 95° C. for 10 minutes, followed by 95° C. for 15 seconds and 60° C. for 1 minute for 40 cycles. Size and purity of the amplification products were verified on a 20% PAA gel. The CT (threshold cycle) is defined as the number of cycles required for the fluorescence signal to exceed the detection threshold. The expression of the target gene relative to the housekeeping gene was calculated as the difference between the threshold values for the two genes.

The SYBR Green method was applied for quantitative amplification of bovine eNOS mRNA. Primer sequences are given in Table 1. In our analysis with the transcription inhibitor α-amanitin—which blocks RNA polymerase II, whereas RNA polymerase I is insensitive to this toxin (Lindell T J et al. Specific inhibition of nuclear RNA polymerase II by alpha-amanitin. Science. 1970; 170: 447-449)—the inventors standardized eNOS mRNA expression to RP29 (ribosomal protein 29) gene as housekeeping gene. Thermal cycling conditions were identical to the TaqMan PCR.

TABLE 1
Sequences of oligonucleotides used in RT-PCR

| | |
|---|---|
| Oligonucleotide | Sequences |

| | |
|---|---|
| Human eNOS-F | 5 ′-GGCATCACCAGGAAGAAGACC-3′ |
| Human eNOS-R | 5 ′-TTCACTCGCTTCGCCATCA-3′ |
| Human eNOS (TaqMan probe) | 5 ′-TAAAGAAGTGGCCAACGCCGTGAAGATCT-3 |
| Human HPRT-F | 5 ′-AGTCTGGCTTATATCCAACACTTCG-3′ |
| Human HPRT-R | 5 ′-GACTTTGCTTTCCTTGGTCAGG-3′ |
| Human HPRT (TaqMan probe) | 5 ′-TTTCACCAGCAAGCTTGCGACCTTGA -3′ |
| Bovine eNOS-F | 5 ′-TTTACCATAAGAGACTGGACCAGAAGTT-3′ |
| Bovine eNOS-R | 5 ′-ATTGACAGCACTGGCTTAGGCA-3′ |
| Bovine HPRT-F | 5 ′-GCTATAAGTTCTTTGCCGACCTGTT-3′ |
| Bovine HPRT-R | 5 ′-TTCTGTTCAGTGCTTTGATGTAATCC-3′ |
| Bovine 28S rRNA-F | 5′-AGTAGCTGGTTCCCTCCGAAGT -3′ |
| Bovine 28S rRNA-R | 5′-TTGCGAGAGCGCCAGCTAT -3′ |

Method 4: Measurement of eNOS Activity

eNOS activity was assessed by the formation of L-[³H]citrulline from L-[³H]arginine after separation of the amino acids by cation exchange chromatography. Endothelial cells were washed with PBS and lysed in the same extraction buffer as for Western blot analysis. The inventors added 50 μg of total protein to the reaction mixture containing 50 mmol/L Hepes pH 7.4, 0.1% Triton X-100, 1 mmol/L EDTA, 1.25 mmol/L CaCl₂, 1 mmol/L DTT, 1 μmol/L FAD, 15 μmol/L BH₄, 1 mmol/L NADPH, and 1 μCi L-[³H]arginine. Incubation was performed for 30 minutes at 37° C. Reactions were terminated by adding 0.5 ml of ice-cold Dowex (Na⁺ form). L-[³H]citrulline was separated from L-[³H]arginine by Dowex chromatography, and L-[³H]citrulline formation was quantified by liquid scintillation counting. The inventors subtracted the values obtained from samples incubated without cell extracts. The inventors added 1 mmol/L nitro-L-arginine methyl ester (L-NAME) in some reactions to prove the specificity of the reaction. In some samples, CaCl₂ was omitted from the reaction buffer to exclude any contribution of inducible NO synthase (iNOS).

Method 5: Vasorelaxation Studies

Thoracic aortas from male Wistar rats were rapidly excised, cleaned of connective tissue, and cut into rings 2 to 3 mm in length for organ-chamber experiments. Rings were incubated with MG132 100 nmol/L, 250 nmol/L, or solvent, or TNF-α (250 pg/ml) in the presence and absence of 250 nM MG132, for 48 hours in MEM at 37° C. containing 50 U/mL penicillin, 50 μg/mL streptomycin, 0.1% BSA, and 1 μg/mL polymyxin B. The rings were then mounted on platinum hooks in 10 ml jacketed organ baths containing modified Krebs-Henseleit solution (composition in mmol/L: NaCl 144, KCl 5.9, CaCli 1.6, MgSO⁴⁻ 1.2, KH₂PO₄⁻ 1.2, NaHCO₃⁻ 25, and D-glucose 11.1) and diclofenac 1 μmol/L. Tension was gradually adjusted to 2 g over 1 hour. The solution in the bath was kept at 37° C. with a gas mixture of 95% CO₂ and 5% O₂. Following equilibration and submaximal precontraction with phenylephrine (0.05 μmol/L), relaxation to increasing concentrations (1 nmol/L to 1 μmol/L) of the endothelium-dependent vasodilator acetylcholine was performed to obtain cumulative concentration-response curves. Selected studies were conducted in rings treated with L-NAME (1 mmol/L) before phenylephrine exposure. Maintenance of smooth-muscle integrity following incubation with the proteasome inhibitor or solvent was confirmed by evaluation of endothelium-independent vasodilation to papaverine (1 nmol/L to 1 μmol/L).

Results

1. Summary

Bovine pulmonary artery endothelial cells (CPAE cells) were treated with the proteasome inhibitor MG132. At 50-250 nmol/L, MG132 increased mRNA as well as protein levels of eNOS, in a dose-dependent manner. Comparable results were obtained with another specific proteasome inhibitor, MG262, whereas the non-proteasomal cathepsin inhibitor ALLM had no effect. Correspondingly, eNOS activity was increased up to 2.8-fold (MG132 100 nmol/L, 48 hours). Similar results were obtained with human umbilical vein endothelial cells (HU-VECs). Incubation of rat aortic rings with MG132 (48 hours) significantly enhanced endothelial-dependent vasorelaxation to acetylcholine. Interestingly, single low-dose MG132 treatment (100 nmol/L) induced long-term effects in CPAE cells, with increases of eNOS protein and enzyme activity up to 10 days. The efficacy of proteasome inhibition was evidenced by Western blots demonstrating accumulation of poly-ubiquitinylated proteins. α-amanitin, an inhibitor of RNA polymerase II, reduced overall eNOS mRNA without influencing proteasome inhibitor-induced elevation of eNOS mRNA suggesting that proteasome inhibition upregulates eNOS by enhancing eNOS mRNA stability.

These results indicate that low-dose inhibition of the proteasome is a valid approach to enhance eNOS expression and activity with potential therapeutic implications.

2. Upregulation of eNOS Protein and mRNA Levels by Proteasome Inhibition

To investigate the effects of proteasome inhibition on eNOS expression, CPAE cells were incubated with the peptide aldehyde proteasome inhibitor MG132. Dose-response experiments revealed that low concentrations of MG132 (50 to 250 nmol/L) gradually increased eNOS protein levels. Higher concentrations of the proteasome inhibitor, of 500 nmol/L and above, on the other hand, decreased eNOS protein levels (FIG. 1A). Since proteasome inhibitors are known to exert cytotoxic effects, the inventors further investigated whether the CPAE cells were damaged by MG132 in the doses applied. Histological examinations (phalloidin-DAPI double staining) revealed that proteasome inhibition induced a dose-dependent phenotypic morphological change of CPAE cells, resulting in an elongated spindle-shaped phenotype of the cells (FIG. 1B). Doses higher than 250 nmol/L, however, proved to be toxic, as determined by trypan blue exclusion (data not shown). Therefore, all further experiments were performed with low non-toxic MG132 concentrations of ≦250 nmol/L. Time- and dose-dependent experiments revealed that the rise of eNOS protein expression began at 16 hours of incubation with MG132 and then further increased, until 48 hours, with 2.4-fold expression (P<0.05) occurring at the concentration of 100 nmol/L (FIG. 1C). This upregulation was obtained after incubation of the cells with a single dose of MG132. To demonstrate that eNOS upregulation is specific to inhibition of the proteasome, the inventors repeated the experiments with another proteasome inhibitor, MG262, a boronic-acid derivative of MG132. Indeed, MG262 in equipotent doses also induced upregulation of eNOS protein to an extent similar to that caused by MG132 (FIG. 1D), whereas ALLM, a non-proteasomal cathepsin inhibitor, failed to upregulate eNOS (data not shown). In addition, the effect of proteasome inhibition was assessed in human cells (HUVECs), for which similar upregulation of eNOS protein expression became evident (FIG. 1E).

In the next step the inventors demonstrated the efficacy of proteasome inhibition with the very low concentrations of MG132 used in our experiments: doses from 50 to 250 nmol/L that increased eNOS expression also led to accumulation of poly-ubiquitinylated proteins. On the other hand, 20 nmol/L of MG132, which did not affect eNOS expression, had no proteasomal inhibitory influence (FIG. 2). Similar results were obtained when the inventors used corresponding doses of MG262, whereas the non-proteasomal cathepsin inhibitor ALLM again failed to inhibit the proteasome (data not shown).

Then, eNOS mRNA levels were measured in CPAE cells. Dose-dependent upregulation of eNOS mRNA was found after incubation with MG132, beginning at 16 hours (data not shown) and reaching a maximum—6.5 times the control level—at 24 hours (100 nmol/L MG132, P<0.05) (FIG. 3A, B). Similar results were obtained with HUVECs (data not shown). To further elucidate whether these increased eNOS mRNA levels may be attributed to increased transcriptional activity or to enhanced mRNA stability, CPAE cells were treated with the transcriptional inhibitor α-amanitin. Administration of α-amanitin reduced overall eNOS mRNA levels without eliminating the increase in mRNA levels induced by MG132 (FIG. 3C). The inventors consequently suggest that proteasome inhibition induces increased eNOS mRNA levels through enhancement of eNOS mRNA stability.

The protective effect that proteasome inhibitors have on eNOS-expression can be seen in FIG. 9 which shows the results of a 48 hours treatment of human umbilical vein endothelial cells (HUVECs) with either DMSO alone as a control and with increasing concentrations of TNF-α (1 ng/ml, 2.5 ng/ml and 5 ng/ml) in the presence and absence of 70 nM MG132. The level of eNOS mRNA expression shown with DMSO is arbitrarily taken to be 1.0, and the other values are put in relation thereto. It can be seen from the experiments with TNF-α alone that increasing concentrations of TNF-α lead to a reduction and almost abolition of mRNA expression at high concentrations of TNF-α, thus showing a damaging effect of TNF-α on eNOS-mRNA expression. In the presence of 70 nM MG132, this harmful effect of TNF-α is reversed and compensated for, resulting in a substantially increased expression of eNOS mRNA when compared to the respective treatment without proteasome inhibitor. This clearly shows the prevention of TNF-α-induced eNOS-suppression by proteasome inhibitors. It is thus a strong indication for a strong protective effect of proteasome inhibitors.

3. Increased eNOS Enzymatic Activity and Improved Endothelial Function by Proteasome Inhibition

The inventors next posed the question whether the increased eNOS protein content observed after proteasome inhibition has a functional consequence in terms of enhanced enzyme activity. The inventors accordingly measured eNOS catalytic activity in protein extracts by conversion of L-arginine to L-citrulline. In parallel with the higher eNOS protein content after MG132 treatment, eNOS activity increased in a dose-dependent manner, beginning at 24 hours and achieving a 2.8-fold increase at 48 hours (100 nmol/L MG132, P<0.05) (FIG. 4 A, B).

To further elucidate the functional importance of eNOS regulation by the proteasome, the impact of proteasome inhibition on the vascular reactivity of isolated rat aortic-ring preparations was investigated in organ-chamber experiments (n=6-8 per group). As shown in FIG. 5A, the endothelium-dependent relaxant responsiveness of phenylephrine-precontracted rings to the endothelium-dependent vasodilator acetylcholine was significantly enhanced after MG132 pretreatment (100 and 250 nmol/L, 48 hours) in a dose-dependent manner: maximum relaxation in MG132-pretreated rings (250 nmol/L): 89.4±5.7%, versus 54.6±5.8% for vehicle-treated rings (P<0.05). The NO synthase inhibitor L-NAME completely abolished acetylcholine-induced vasodilation (data not shown). Endothelial-independent vasorelaxation in response to papaverine remained unaffected by proteasome inhibition (FIG. 5B).

4. Long-Term Upregulation of eNOS Expression and Activity

In order to assess how long a single dose of the proteasome inhibitor MG132 is able to upregulate eNOS expression and activity, the experiments were extended up to 10 days: the inventors found still elevated eNOS mRNA after 7 days (data not shown), enhanced protein, as well as enzyme activity up to 10 days following proteasome inhibition (100 nmol/L MG132) in CPAE cells (FIG. 6A, B). Interestingly, long-term eNOS upregulation was observed despite the fact that accumulation of poly-ubiquitinylated proteins—which indicate the efficacy of proteasome inhibition—were detectable for no longer than three days (FIG. 6C).

5. Protective Effect of Proteasome Inhibitors on Endothelial Function

To directly show a protective effect of proteasome inhibitors on endothelial function, which effect goes beyond the aforementioned increase in eNOS enzymatic activity, upregulation of eNOS expression etc., experiments were performed that were aimed at showing that proteasome inhibitors prevent damaging agents like cytokines, e.g. TNF-α, oxLDL or LPS, to exert their damaging effects on the endothelium. In order to measure endothelial function, vasoreactivity of phenylephrine-precontracted rat aortic rings was tested by means of acetylcholine (FIGS. 7 and 8). To simulate a damaging effect, aortic rings were incubated for 48 hours with damaging agents (TNF-α, oxLDL, LPS), and thereafter the resulting damaging effect on endothelial function was measured. FIG. 7 shows a summary of a series of experiments performed with TNF-α (250 pg/ml). Compared to the control (DMSO as solvent) the acetylcholine-dependent vasodilatation is significantly reduced in the aortic rings preincubated with TNF-α. In the figure a 100% contraction corresponds to the state in which the aortic rings were directly after the phenylephrine-precontraction. 0% contraction corresponds to a fully relaxed/vasodilatated state as induced by higher concentrations of acetylcholine on the DMSO-control. For testing a possible protective effect of proteasome inhibitor, aortic rings were incubated with both TNF-α and the proteasome inhibitor MG132. FIG. 7 shows that the damage of the endothelium, which damage is induced by the TNF-α, can be significantly reduced, and the vasoreactivity can be significantly improved by application of proteasome inhibitors. FIG. 8 shows a single experiment of FIG. 7 as an example (original traces).

Thus, proteasome inhibitors display significant protective effects with regard to a prevention/reduction/regression of endothelial dysfunction and are thus prime candidates for the manufacture of a medicament for the prevention, onset therapy, acute therapy and/or regression of diseases associated with endothelial dysfunction.

1. Use of at least one proteasome inhibitor for the manufacture of a medicament for the prevention, onset therapy, acute therapy and/or regression of diseases associated with endothelial dysfunction.

2. Use according to claim 1, wherein the diseases associated with endothelial dysfunction are non-insulin related diseases.

3. Use according to claim 1, wherein the endothelial dysfunction is associated with atherosclerosis, in particular coronary sclerosis and coronary artery disease.

4. Use according to claim 1, wherein the endothelial dysfunction is associated with heart failure.

5. Use according to claim 1, wherein the endothelial dysfunction is associated with diseases selected from the group comprising ischemic diseases such as peripheral arterial occlusive disease, e.g. critical leg ischemia, myocardial infarction and ischemic diseases of organs, e.g. of the kidney, spleen, brain, and lung.

6. Use according to claim 1, wherein the proteasome inhibitor is selected from a group comprising:
a) naturally occurring proteasome inhibitors comprising:
peptide derivatives which have a C-terminal expoxy keton structure, β-lacton-derivatives, aclacinomycin A, lactacystin, clastolactacystein;
b) synthetic proteasome inhibitors comprising:
modified peptide aldehydes such as N-carbobenzoxy-L-leucinyl-L-leucinyl-L-leucinal (also referred to as MG132 or ZLLL), or the boric acid derivative of MG232, N-carbobenzoxy-Leu-Nva-H (also referred to as MG115), N-acetyl-L-leucinyl-L-leucinyl-L-norleucinal (also referred to as LLnL), N-carbobenzoxy-Ile-Glu(OBut)-Ala-Leu-H (also referred to as PS1) [SEQ ID NO: 1];
peptides comprising:
an α,β,-epoxyketone-structure, vinyl-sulfones such as, carbobenzoxy-L-leucinyl-L-leucinyl-L-leucin-vinyl-sulfon or, 4-hydroxy-5-iodo-3-nitrophenylacetyl-L-leucinyl-L-leucinyl-L-leucin-vinyl-sulfon (NLVS);
d) Glyoxal- or boric acid residues such as: pyrazyl-CONH(CHPhe)CONH(CHisobutyl)B(OH)₂ and dipeptidyl-boric-acid derivatives;
e) Pinacol-esters such as: benzyloxycarbonyl(Cbz)-Leu-leuboro-Leu-pinacol-ester.

7. Use according to claim 1, wherein the proteasome inhibitor is selected from a group comprising PS-314 as a peptidyl-boric-acid derivative which is N-pyrazinecarbonyl-L-phenylalanin-L-leucin-boric acid (C₁₉H₂₅BN₄O₄); PS-519 as a β-lacton- and a lactacystin-derivative which is 1R-[1S,4R,5S]-1-(1-Hydroxy-2methylpropyl)-4-propyl-6-oxa-2azabicyclo[3.2.0]heptane-3,7-dione (C₁₂H₁₉NO₄); PS-273 (morpholin-CONH—(CH-naphthyl)-CONH—(CH-isobutyl)-B(OH)₂) and its enantiomer; PS-293; PS-296 (8-quinolyl-sulfonyl-CONH—(CH-napthyl)-CONH(—CH-isobutyl)-B(OH)₂); PS-303 (NH₂(CH-naphthyl)-CONH—(CH-isobutyl)-B(OH)₂; PS-321 as (morpholin-CONH—(CH-naphthyl)-CONH—(CH-phenylalanin)-B(OH)₂); PS-334 (CH₃-NH-(CH-naphthyl-CONH—(CH-Isobutyl)-B(OH)₂); PS-325 (2-quinol-CONH—(CH-homo-phenylalanin)-CONH—(CH-isobutyl)-B(OH)₂; PS-352 (phenyalanin-CH₂—CH₂—CONH—(CH-isobutyl)I—B(OH)₂; PS-383 (pyridyl-CONH—(CHₚF-phenylalanin)-CONH—(CH-isobutyl)-B(OH)₂); PS-341; and PS-1 Z-Ile-Glu(OtBu)-Ala-Leu-CHO [SEQ ID NO: 11]; PS-2 [Benzyloxycarbonyl)-Leu-Leu-phenylalaninal or Z-LLF-CHO or Z-Leu-Leu-Phe-CHO PS-1; PS-519 as a β-lacton- and a lactacystin-derivative which is 1R-[1S,4R,5S]-1-(1-Hydroxy-2methylpropyl)-4-propyl-6-oxa-2azabicyclo[3.2.0]heptane-3,7-dione (C₁₂H₁₉NO₄); epoxomicin (C₂₈H₈₆N₄O₇) and eponemycin (C₂₀H₃₆N₂O₅).

8. Use according to claim 1, wherein the proteasome inhibitor is selected from a group comprising a peptide aldehyde, a peptide boronate, a peptide vinylsulfone, a peptide epoxyketone, a lactacystin, a peptide α-ketonaldehyde, an α-ketoamide, an indanonpeptide, a polyalkylenaldehyde, a polyphenol such as cathechin-3-gallate.

9. Use according to claim 1, wherein the proteasome inhibitor is selected from a group comprising Z-Leu-Leu-Leu-al (MG132), Z-Ile-Glu(OtBu)-Ala-Leu-al (PS-1) [SEQ ID NO: 1], CEP1612, pyrazylcarbonyl-Phe-Leu-boronate (PS-341), dansyl-Phe-Leu-boronate (DFLB), morpholinonaphthylalanin-Leu-boronate (MG273), NIP-Leu₃-vinylsulfone (NLVS), Tyr-Leu₃-VS [SEQ ID NO: 2], NIP-Leu-Leu-Asn-VS, Ada-Tyr-Ahx₃-Leu₃-VS [SEQ ID NO: 3], Ada-Lys(bio)-Ahx₃-Leu₃-VS [SEQ ID NO: 4], Ac(Me)-Ile-Ile-Thr-Leu-EX (epoxomicin) [SEQ ID NO: 5], dihydroeponemycin, lactacystin, clasto-lactacystin-beta-lacton (omuralid), PS-519, Ac-Leu-Leu-Nle-al (ALLN), 3,4-dichloroisocoumarin (DCl), 4-(2-aminoethyl)-bezolsulfonylfluorid (pefablock SC), TMC-95-A, gliotoxin, (−)epigallocatechin-3-gallate (EGCG), ritonavir, lovastatin, aclacinomicin A (aclarubicin), cyclosporin, wherein Z represents benzyl oxycarbonyl, all represents aldehyde, VS represents vinylsulfone, NIP represents 3-nitro-4-hydroxy-5-iodophenylacetate, and bio represents biotin.

10. Use according to claim 1, wherein the proteasome inhibitor interferes with gene expression of at least one component of the proteasome complex.

11. Use according to claim 10, wherein the proteasome inhibitor interfering with proteasomal gene expression is selected from a group comprising antisense RNA, double stranded RNA and oligonucleotides hybridising with a DNA sequence encoding at least one component of the proteasome complex.

12. Use according to claim 10, wherein the proteasome inhibitor interfering with proteasomal gene expression is selected from a group comprising a knock out construct.

13. Use of at least one proteasome inhibitor for the manufacture of a medicament for the prevention, onset therapy, acute therapy and/or regression of a disease, wherein the proteasome inhibitor dose provided to a patient in need is in the nmol range.

14. Use according to claim 13, wherein the disease is associated with endothelial dysfunction.

15. Use according to claim 13, wherein the disease associated with endothelial dysfunction is a non-insulin related disease.

16. Use according to claim 13, wherein the endothelial dysfunction is associated with atherosclerosis, in particular coronary sclerosis and coronary artery disease.

17. Use according to claim 13, wherein the endothelial dysfunction is associated with heart failure.

18. Use according to claim 13, wherein the endothelial dysfunction is associated with diseases selected from the group comprising ischemic diseases such as peripheral arterial occlusive disease, e.g. critical leg ischemia, myocardial infarction and ischemic diseases of organs, e.g. of the kidney, spleen, brain, and lung.

19. Use according to claim 13, wherein the proteasome inhibitor is selected from a group comprising:
a) naturally occurring proteasome inhibitors comprising:
peptide derivatives which have a C-terminal expoxy keton structure, β-lacton-derivatives, aclacinomycin A, lactacystin, clastolactacystein;
b) synthetic proteasome inhibitors comprising:
modified peptide aldehydes such as N-carbobenzoxy-L-leucinyl-L-leucinyl-L-leucinal (also referred to as MG132 or zLLL), or the boric acid derivative of MG232, N-carbobenzoxy-Leu-Nva-H (also referred to as MG115), N-acetyl-L-leucinyl-L-leucinyl-L-norleucinal (also referred to as LLnL), N-carbobenzoxy-Ile-Glu(OBut)-Ala-Leu-H (also referred to as PS-1) [SEQ ID NO: 1];
c) peptides comprising:
an α,β,-epoxyketone-structure, vinyl-sulfones such as, carbobenzoxy-L-leucinyl-L-leucinyl-L-leucin-vinyl-sulfon or, 4-hydroxy-5-iodo-3-nitrophenylacetyl-L-leucinyl-L-leucinyl-L-leucin-vinyl-sulfon (NLVS);
d) Glyoxal- or boric acid residues such as: pyrazyl-CONH(CHPhe)CONH(CHisobutyl)B(OH)₂ and dipeptidyl-boric-acid derivatives;
e) Pinacol-esters such as: benzyloxycarbonyl(Cbz)-Leu-leuboro-Leu-pinacol-ester.

20. Use according to claim 13, wherein the proteasome inhibitor is selected from a group comprising PS-314 as a peptidyl-boric-acid derivative which is N-pyrazinecarbonyl-L-phenylalanin-L-leucin-boric acid (C₁₉H₂₅BN₄O₄); PS-519 as a β-lacton- and a lactacystin-derivative which is 1R-[1S,4R,5S]-1-(1-Hydroxy-2methylpropyl)-4-propyl-6-oxa-2azabicyclo[3.2.0]heptane-3,7-dione (C₁₂H₁₉NO₄); PS-273 (morpholin-CONH-—(CH-naphthyl)-CONH—(CH-isobutyl)-B(OH)₂) and its enantiomer; PS-293; PS-296 (8-quinolyl-sulfonyl-CONH—(CH-napthyl)-CONH(—CH-isobutyl)-B(OH)₂); PS-303 (NH₂(CH-naphthyl)-CONH—(CH-isobutyl)-B(OH)₂; PS-321 as (morpholin-CONH—(CH-naphthyl)-CONH—(CH-phenylalanin)-B(OH)₂); PS-334 (CH₃—NH—(CH-naphthyl-CONH—(CH-isobutyl)-B(OH)₂); PS-325 (2-quinol-CONH—(CH-homo-phenylalanin)-CONH—(CH-isobutyl)-B(OH)₂; PS-352 (phenyalanin-CH₂—CH₂—CONH—(CH-isobutyl)I—B(OH)₂; PS-383 (pyridyl-CONH—(CHₚF-phenylalanin)-CONH—(CH-isobutyl)-B(OH)₂); PS-341; and PS-1 Z-Ile-Glu(OtBu)-Ala-Leu-CHO [SEQ ID NO: 1]; PS-2 [Benzyloxycarbonyl)-Leu-Leu-phenylalaninal or Z-LLF—CHO or Z-Leu-Leu-Phe-CHO PS-1; PS-519 as a β-lacton- and a lactacystin-derivative which is 1R-[1S,4R,5S]-1-(1-Hydroxy-2methylpropyl)-4-propyl-6-oxa-2azabicyclo[3.2.0]heptane-3,7-dione (C₁₂H₁₉NO₄); epoxomicin (C₂₈H₈₆N₄O₇) and eponemycin (C₂₀H₃₆N₂O₅).

21. Use according to claim 13, wherein the proteasome inhibitor is selected from a group comprising a peptide aldehyde, a petipde boronate, a peptide vinylsulfone, a peptide epoxyketone, a lactacystin, a peptide α-ketonaldehyde, an α-ketoamide, an indanonpeptide, a polyalkylenaldehyde, a polyphenol such as cathechin-3-gallate.

22. Use according to claim 13, wherein the proteasome inhibitor is selected from a group comprising Z-Leu-Leu-Leu-al (MG132), Z-Ile-Glu(OtBu)-Ala-Leu-al (PS-1) [SEQ ID NO: 1], CEP1612, pyrazylcarbonyl-Phe-Leu-boronate (PS-341), dansyl-Phe-Leu-boronate (DFLB), morpholinonaphthylalanin-Leu-boronate (MG273), NIP-Leu₃-vinylsulfone (NLVS), Tyr-Leu₃-VS [SEQ ID NO: 2], NIP-Leu-Leu-Asn-VS, Ada-Tyr-Ahx₃- Leu₃-VS [SEQ ID NO: 3], Ada-Lys(bio)-Ahx₃-Leu₃-VS [SEQ ID NO: 4], Ac(Me)-Ile-Ile-Thr-Leu-EX (epoxomicin) [SEQ ID NO: 5], dihydroeponemycin, lactacystin, clasto-lactacystin-beta-lacton (omuralid), PS-519, Ac-Leu-Leu-Nle-al (ALLN), 3,4-dichloroisocoumarin (DCl), 4-(2-aminoethyl)-bezolsulfonylfluorid (pefablock SC), TMC-95-A, gliotoxin, (−)epigallocatechin-3-gallate (EGCG), ritonavir, lovastatin, aclacinomicin A (aclarubicin), cyclosporin, wherein Z represents benzyl oxycarbonyl, all represents aldehyde, VS represents vinylsulfone, NIP represents 3-nitro-4-hydroxy-5-iodophenylacetate, and bio represents biotin.

23. Use according to claim 13, wherein the proteasome inhibitor is MG132.

24. Use according to claim 13, wherein the proteasome inhibitor interferes with gene expression of at least one component of the proteasome complex.

25. Use according to claim 13, wherein the proteasome inhibitor interfering with proteasomal gene expression is selected from a group comprising antisense RNA, double stranded RNA and oligonucleotides hybridising with a DNA sequence encoding at least one component of the proteasome complex.

26. Use according to claim 13, wherein the proteasome inhibitor interfering with proteasomal gene expression is selected from a group comprising a knock out construct.
